(19) United States
(12) Patent Application Publication
(10) Pub. No.: US 2005/0009057 A1
(43) Pub. Date: Jan. 13, 2005

(54) Assay for detecting the presence of processing inhibitory antibodies against the Apical Membrane Antigen-1 of Plasmodium falciparum in biological samples

(76) Inventors: Sheetij Dutta, Silver Spring, MD (US); David E. Lanar, Takoma Park, MD (US); John David Hannes, Chevy Chase, MD (US)

Correspondence Address: U. S. Army Medical Research and Materiel Command;ATTN: MCMR-JA (Ms. Elizabeth Arwine-PATENT ATTY), 504 Scott Street, Fort Detrick, MD, 21702-5012, US

(21) Appl. No.: 10/839,558

(22) Filed: May 5, 2004

Related U.S. Application Data
(60) Provisional application No. 60/468,007, filed on May 5, 2003.
(60) Provisional application No. 60/476,399, filed on Jun. 6, 2003.

(51) Int. Cl. C12Q 1/68; G01N 33/53; G01N 33/569; C12P 21/06

(52) U.S. Cl. 435/6

(57) ABSTRACT

In this application is described a method for determining the presence or absence of functional anti-AMA-1 invasion-inhibitory antibodies in a sample. This method can serve as a correlate for immunity of a P. falciparum AMA-1-based vaccine or as a correlate for immunity to P. falciparum by natural exposure if that immunity was induced by invasion inhibitory antibodies against the AMA-1 protein.

This application claims the benefit for priority under 35 U.S.C. §119(e) from Provisional Application Ser. No. 60/468,007 filed on May 5, 2003 and 60/476,399 filed on Jun. 6, 2003.

An assay for detecting the presence of processing inhibitory antibodies against the Apical Membrane Antigen-1 of Plasmodium falciparum in biological samples.

### INTRODUCTION

Plasmodium falciparum is the leading cause of malaria morbidity and mortality. The World Health Organization estimates that approximately 200 million cases of malaria are reported yearly, with 3 million deaths (World Health Organization, 1997, Wkly. Epidemiol. Rec. 72:269-276). Although, in the past, efforts have been made to develop effective controls against the mosquito vector using aggressive applications of pesticides, these efforts ultimately led to the development of pesticide resistance. Similarly, efforts at treatment of the disease through anti-parasitic drugs led to parasite drug-resistance. As the anti-vector and anti-parasite approaches have failed to control the spread of the disease, efforts became focused on malaria vaccine development as an effective and inexpensive alternative approach.

However, the complex parasitic life cycle has further confounded the efforts to develop efficacious vaccines for malaria. The parasite's life cycle is divided between the mosquito-insect host and the human host. While in the human host, it passes through several developmental stages in different cellular environments, i.e. the liver stages and the red blood stages. Although conceptually simple, in reality the problems that must be considered when designing subunit vaccines for malaria are great. A high degree of developmental stage specificity, antigenic variation and antigen polymorphisms have been reported in most of the promising vaccine candidates. There is a need to understand the vital and conserved pathways involved in the invasion and intra-cellular development of the parasite in order to develop a vaccine that is effective globally. One such conserved pathway appears to be the need for stage-specific processing of important malarial antigens.

Proteases play an important role in the process of host cell invasion and intracellular development of protozoan parasites (Proteases of Infectious Agents, 1991 eds. Dunn, B. M. Academic Press). Parasite proteases assist invasion directly by modifying host RBC membrane or indirectly by proteolytic processing of other merozoite proteins, which in turn are involved in invasion (Blackman, M. J., 2000, Curr. Drug Targets 1, 59-83). The best known example of the importance of protease action during invasion is the processing of Merozoite surface protein-1 (MSP-1) of Plasmodium falciparum, an important vaccine candidate for malaria. MSP-1 is synthesized as a ˜200 kDa protein. As a result of several proteolytic cleavages during merozoite development, the ˜200 kDa protein is processed to a 19 kDa merozoite bound molecule (MSP-1₁₉) which is believed to be of functional significance during invasion (Holder, A. A. et al., 1999, Parasitologia 41, 409-414). Antibodies against MSP-1₁₉, which block invasion of merozoites into RBC, have also been shown to interrupt the crucial proteolytic step that gives rise to MSP-1₁₉ (Blackman, M. J. et al., 1994, J. Exp. Med. 180, 389-393).

Apical Membrane Antigen-1 is another important P. falciparum protein (PfAMA-1) being actively considered for vaccine development (Peterson, M. G. et al., 1989, Mol. Cell. Biol. 9, 3151-3154). AMA-1 is a type I transmembrane protein found on all the malaria parasites studied so far, human (Peterson, M. G. et al., 1989, supra; Cheng and Saul, 1994, Mol. Biochem. Parasitol. 65, 183-187), primate (Dutta et al., 1995, Mol. Biochem. Parasitol. 73, 267-270; Waters et al., 1990, J. Biol. Chem. 265, 17974-17979), or rodent (Kappe and Adams, 1996, Mol. Biochem. Parasitol. 78, 279-283). Amino acid sequence alignment shows the presence of 16 inter-species conserved cysteines, which are known to form 8 disulphide bonds (Hodder et al., 1996, J. Biol. Chem. 271, 2946-29452); the tertiary structure of AMA-1 resulting from the disulphide bond formation is critical for inducing a protective antibody response (Anders et al., 1998, Vaccine 16, 240-247).

Like MSP-1, PfAMA-1 is also synthesized as a precursor protein of 83 kDa (PfAMA-1₈₃) (Peterson et al, 1989, supra; Narum, D. L. and Thomas, A. W., 1994, Mol. Biochem. Parasitol. 67, 59-68). PfAMA-183 is localized in the apical complex (Peterson et al, 1989, supra; Narum and Thomas, 1994, supra; Crewther, P. E. et al., 1990, Expt. Parasitol. 70, 193-206; Healer, J. et al., 2002, Infect. Immun. 70, 5751-5758; Kocken, C. H. et al., 1998, J. Biol. Chem. 273, 15119-15124) of the merozoite within the infected erythrocyte. At the time of schizont rupture, PfAMA-1₈₃ is processed to a 66 kDa form (PfAMA-1₆₆) by the removal of a short N-terminal prosequence (Narum and Thomas, 1994, supra; Howell, S. A. et al., 2001, J. Biol. Chem. 276, 31311-31320). At or around schizont rupture and merozoite invasion, PfAMA-1₆₆ translocates from within the apical complex to the surface of the merozoite (Narum and Thomas, 1994, supra; Healer et al., 2002, supra; Kocken, C. H. et al., 1998, supra). Once on the surface PfAMA-1₆₆ is circumferentially redistributed and undergoes two C-terminal cleavages (either sequentially or independently), giving rise to 48 and 44 kDa soluble forms (PfAMA-1₄₈, PfAMA-1₄₄) which are normally shed into the culture medium (Narum and Thomas, 1994, supra; Howell et al., 2001, supra; Howell, S. A., et al., 2003, J. Biol. Chem. 278, 23890-23898). Processed forms containing the C-terminal end of PfAMA-1 have been detected on the ring forms (Narum and Thomas, 1994, supra; Howell et al., 2001, supra;). Although the exact relationship between processing, translocation, redistribution and shedding events of AMA-1 is not clear, their timing suggests involvement in merozoite invasion. Recombinant P. falciparum AMA-1 protein induces anti-parasitic antibodies, which inhibit parasite growth in vitro (Hodder, A. N. et al., 2001, Infect. Immun. 69, 3286-3294; Kennedy, M. C. et al., 2002, Infect. Immun. 70, 6948-6960) and protect immunized animals against parasite challenge in vivo (Stowers, A. W. et al., 2002, Infect. Immun. 70, 6961-6967). We have manufactured GMP-grade recombinant AMA-1 protein from the P. falciparum 3D7 clone (Dutta, S. et al., 2002, Infect. Immun. 70, 3101-3110) for testing this protein as a malaria vaccine. Although this protein has not been tested in a non-human primate challenge model for P. falciparum malaria (due to the inability of the 3D7 parasite to infect monkeys), antibodies to this protein effectively inhibit invasion of the parasites in vitro (Dutta, S. et al., 2002, supra).

### SUMMARY OF THE INVENTION

This application describes the first observations of the effects of invasion-inhibitory anti-AMA-1 antibodies on parasite AMA-1 processing and redistribution. Until recently the only known assay to measure the presence of inhibitory antibodies in vaccinated individuals was the in vitro growth or invasion inhibition assay. We have discovered that: (1) Bivalent IgG and monovalent Fab fragments that block invasion cause significant inhibition of PfAMA-1₆₆ processing. (2) bivalent IgG can cross-link two soluble forms of AMA-1, i.e. PfAMA-1₄₈ and PfAMA-1₄₄ on the merozoite. (3) Bivalent, polyclonal antibodies can inhibit the circum-merozoite redistribution and shedding of PfAMA-1. Our data suggests that antibodies to AMA-1 can affect the processing of native AMA-1 at concentrations achievable following vaccination (Dutta et al. Proc., 2003, Natl Acad Sci USA, 100, 12295-300). . . .

In this application we disclose a processing inhibition assay which can be used to determine the processing inhibitory activity of anti-AMA-1 immune reagents and chemical inhibitors. The assay described here is sensitive to the strain specificity seen in the in vitro growth or invasion inhibition assay (GIA). We believe that assays based on this invention will be predictive of a AMA-1 based protective response in immunized animals and in humans and will serve as a reliable correlate of AMA based immunity whether inferred by natural infection or immunization.

Therefore, it is an object of the present invention to provide a general method for detecting the presence or absence of anti-AMA-1 processing inhibitory antibodies in a sample. Evidence is also presented to show that the processing inhibitory activity of anti-AMA-1 antibodies directly correlates with its ability to inhibit invasion. The said method comprises incubating the sample with the Plasmodium falciparum parasite's schizont stage. Known standard sera of high processing inhibitory activity or growth inhibitory activity can serve as positive controls and sera with no or low processing inhibitory activity can serve as a negative control. Following schizont rupture, the merozoites will be harvested and AMA-1 specific bands will be analyzed and quantitated by any method known in the art, for example, by FACS, ELISA or by western blot. For example, using densitometric analysis on the western blots the relative quantities of PfAMA-1 specific bands will be calculated. It is expected that the following AMA-1 specific bands will be detected. The approximately 83 kDa, approximately 66 kDa, approximately 52 kDa (if polyclonal antibodies are being used), approximately 20 kDa membrane bound form left on the merozoite following the shedding of the soluble forms, and an approximately 10 kDa form left over on the merozoite following the cleavage of the approximately 52 kDa form. Processing inhibitory activities of unknown samples can be calculated and comparisons can be made between the unknown and known standard samples. We routinely observe that the intensity of the residual 66, 52 and 10 kDa bands on the merozoite following rupture, inversely correlates the invasion inhibitory potential of a serum sample. An example of a method to calculate the processing inhibitory activity of a serum from the band intensities (pixel density values) on a western blot for a polyclonal serum is: Percent PfAMA-1₁₀/(PfAMA-1₁₀+PfAMA-1₂₀).

We have also discovered that processing inhibition can also be measured by detecting shed AMA-1 from culture supernatants using a processing inhibition assay as described above, however, instead of harvesting the merozoite pellet the supernatant can be collected, analyzed and quantitated by methods known in the art such as FACS analysis, ELISA or on a western blot. The amount of 44+48 immunoprecipitated can determine whether or not the antibodies are inhibitory, wherein the higher the amount of 44+48 in the supernatant the lower the processing inhibitory activity of the test sample.

It is another object of the invention to provide one or more compositions wherein the composition inhibits P. falciparum AMA-1 processing. Such compositions would include protease inhibitors, anti-AMA-1 antibodies, and other compounds that affect AMA-1 processing.

It is an object of the present invention to provide a method for screening the activity of drugs and compounds aimed to have anti-malarial activity via blocking the AMA-1 processing. An assay comprises incubating a test sample with either recombinant or native AMA-1 of P. falciparum and detecting and quantifying the merozoite bound AMA-1 processing products and intermediates that can be trapped on the merozoites using sub-inhibitory concentrations of antibodies to AMA-1. The compounds may be useful as a therapeutic compound for a subject infected with the parasite.

It is an object of the present invention to provide an assay for inhibition of redistribution of AMA-1 on merozoites. This redistribution event is inhibitied in the presence of polyclonal antibodies against AMA-1, an assay based on the inhibition of redistribution (Dutta et. al. PNAS) may correlate the ability of that serum sample to inhibit parasite invasion of the merozoites into RBC.

It is envisioned that novel AMA-1 DNA constructs can be selected by mutagenesis to provide an AMA-1 protein which can elicit higher quantities of processing inhibitory antibodies upon administration to a subject. Such modified AMA-1 proteins may provide a higher level of protection against parasite infection.

All the objects of the present invention are considered to have been met by the embodiments as set out below.

### BRIEF DESCRIPTION OF THE DRAWINGS

FIG. 1. ANTIBODIES AFFECT THE PROCESSING OF AMA-10N DEVELOPING MEROZOITES: (A) Processing assay with immune serum pool at two dilutions. Each lane represents merozoites released from ˜1.4×10⁵ schizonts. Lane 1, pre-immune (1:10 dilution); lanes 2, 3 post-immune at 1:10 and 1:2500 dilution respectively; lane 4, soluble AMA-1 fragments immuno-precipitated from culture supernatant (representative of ˜5×10⁶ rupturing schizonts, assuming 100% recovery). (B) Processing assay samples corresponding to lane 3 and lane 4 of FIG. 1A run under reduced conditions and immunostained with biotin labeled IgG against reduced and alkylated AMA-1 protein. (C) Immuno-precipitation from culture supernatants of the processing assay containing 1:10 pre-immune (lane 1), 1:10 post-immune (lane 2) and 1:2500 post-immune serum (lane 3). PfAMA-1 specific bands and molecular weight marker positions (Multimark, Invitrogen) are shown with arrows.

FIG. 2. THE EFFECT OF ANTIBODIES ON AMA-1 PROCESSING IS DOSE DEPENDENT: Processing assay showing dose response of an individual inhibitory rabbit serum on AMA-1 processing. Final serum dilutions used in the assay were 2400, 810, 270, 90, 30 and 20.

FIG. 3. THE ASSAY CAN DETECT REAL_TIME PROCESSING OF AMA-1. Effect of anti-AMA-1 antibodies on AMA-1 processing during merozoite maturation and schizont release. Processing assay performed with 1:10 dilution of preimmune or post immune pools. Samples were drawn at 5 time points (T1-T5 corresponding to 0-90% rupture; see results). To rule out immuno-precipitation from culture supernatant, post-immune sera was added to a pre-immune sera containing well (at T5, keeping the final dilution 1:10) and incubation continued at 37° C. for another 30 min (lane-a, post immune sample at T6; lane-b, pre-immune control incubated with the post-immune sera at T6). Lane-c corresponds to the sample in lane-a analyzed for reactivity to AMA-1 specific mAb 4G2dc1.

FIG. 4. PROCESSING ASSAY IS HIGHLY SPECIFIC FOR INVASION INHIBITORY ANTIBODIES (A) Processing assay showing the effects of IgG against reduced and alkylated AMA-1 (IgG-R/A) and refolded AMA-1 (IgG-Ref) proteins. IgG concentration in lanes 1-4 were 0.00035, 0.0035, 0.035 and 0.35 mg/ml, respectively. (B) Processing assay showing the effect of anti-AMA-1 Fab fragments. Lanes 1-5, 0.000014, 0.00014, 0.0014, 0.014, 0.14 mg/ml Fabs, respectively; C, media control. Parallel assay with trapping antibodies (1:2500 post-immune pool) is also shown.

FIG. 5. POLYCLONAL ANTIBODIES INHIBIT THE REDISTRIBUTION OF AMA-10N DEVELOPING MEOZOITES Double immuno-fluorescence image, using a dual cube filter, of free merozoites released in presence of pre-immune (left, 1:10 dilution) or post-immune (right 1:10 dilution) serum. The pre-immune sample was incubated with 1:10 post-immune sera for 1.5 h on ice after rupture. Slides were acetone fixed and AMA-1 was visualized by staining with FITC conjugated anti-rabbit (green fluorescence) and merozoite surface demarcated by staining with P. falciparum MSP-1 specific mAb 5.2 and anti mouse-Phycoerythrin (red fluorescence). Inset shows enlarged view of a single merozoite.

FIG. 6 ANTIBODIES CAN BE USED TO TRAP THE PRODUCTS OF AMA-1 PROCESSING ON MEROZOITES MAKING THIS AN EXCELLENT ASSAY FOR SCREENING DRUGS THAT BLOCK AMA-1 PROCESSING Panel-a. Processing assay in the presence of protease inhibitors. Panel-b. Identical assay performed in the presence of trapping antibodies (1:2500 AMA-1 immune serum pool). (A) Lanes 1-PMSF (200 μM), 2-TLCK (100 μM), 3-TPCK (100 μM), 4-leupeptin (100 μM), 5-chymostatin (100 μM), 6-antipain (100 μM), 7-E64 (10 μM), 8-pepstatin (5 μM), 9-1,10-Phenanthroline (1 μM), 10-EDTA, (1 mM) and 11-EGTA (1 mM), 12-ethanol control, 13-DMSO control, 14-PBS control. (B) Dose response of chymostatin, EDTA and EGTA on AMA-1 processing. Concentrations of inhibitors used from lanes 1-4 were chymostatin: 100, 50, 25, 12.5 μM; EDTA & EGTA: 2, 1, 0.5, 0.25 mM respectively; lane c, DMSO control; lane c′, PBS control. (C) Processing assay showing the effect of 1 mM MgCl₂ or 1 mM CaCl₂ added to reverse the EDTA and EGTA (1 mM each) mediated processing inhibition. Lanes 1, EDTA; 2, EGTA; c, PBS control.

FIG. 7. USING A MIXTURE OF A C-TERMINUS MAB ALONG WITH THE POLYCLONAL SERUM TO DETECT AMA-1 FRAGMENTS ALSO SHOWS THAT ANTIBODIES AGAINST AMA-1 INHIBIT PROCESSING Time course processing inhibition assay. P. falciparum 3D7 schizonts were incubated 37° C., with either pre-immune (1:10 dilution), post-immune (1:2500) or post-immune (1:10) and samples were harvested at four time points T₀=0% rupture, T₁=30%, T₂=50%, T₃=70%, T₄=90% rupture. The samples were run on SDS-PAGE and western blotted. Blot shown in FIG. 8, was probed with a mixture of two biotinylated primary antibodies: polyclonal anti-AMA-1 IgG and monoclonal 28G8dc1. Position of molecular weight marker bands (Multimark, Invitrogen) is represented in kDa on the left, and position of the respective PfAMA-1 bands is shown on the right. NS=Non-specific bands present in uninfected erythrocytes incubated with 1:10 post-immune serum control lane C.

FIG. 8. EFFECT OF ANTIBODIES ON AMA-1 PROCESSING IS DOSE DEPENDENT Effect of serum dilution on the processing of AMA-1: Schizonts were allowed to rupture in the presence of 1:10, 1:00 and 1:1000 dilution of either pre-, or post-immune anti-AMA-1 antisera. Lanes 1, 2-1:10, 100 pre-immune serum; lanes 3, 4, 5-1:10, 100, 1000 dilutions of post-immune sera. Blot shown in FIG. 8 was probed as described in FIG. 7.

FIG. 9. METHOD TO ACCESS THE PROCESSING INHIBITIORY ACTIVITY OF A SERUM SAMPLE AND THAT THIS ACTIVITY CORRELATES INVASION INHIBITORY ACTIVITY Assay for PIA activity: A. Experiment 1, PIA blot: Sera from an immunogenicity study in rabbits immunized with either the 3D7, FVO or a mixture of the two proteins along with adjuvants was used in the study. IgG preparations from rabbit sera immunized with refolded or reduced and alkylated 3D7 antigen were also used in addition to the negative rabbit serum control. Lanes 1-12, rabbit anti-AMA-1 immune sera; 13, adjuvant control serum; 14, No rabbit serum control, 15-18, anti-AMA-1 immune sera; 19, pre-immune control, 20, IgG against refolded protein (3.5 mg/ml), 21, IgG against reduced and alkylated protein (3.5 mg/ml). All sera were tested at 1:10, 1:00 and 1:1000 dilution in a PIA, however, FIG. 10 shows only the 1:100 dilution (best correlation with GIA). Blot was probed as described in FIG. 7. The respective GIA and PIA activity values are also shown. B. Expt 1, correlation plot between GIA and PIA: GIA activity expressed as percent of band intensity of PfAMA-1₁₀/(PfAMA-1₁₀+PfAMA-1₂₀) at 1:100 serum dilution. GIA was carried out at 20% rabbit serum and GIA activity was reported as percent inhibition of invasion compared with adjuvant control sera (Montanide). R² value is also shown. C. Expt 2, PIA blot: Lanes 1, adjuvant control serum; 2-9, immune anti-AMA-1 rabbit serum samples; 10, no rabbit serum control. D. Expt 2, correlation plot between GIA and PIA.

### DETAILED DESCRIPTION

In the description that follows, a number of terms used in recombinant DNA, parasitology and immunology are extensively utilized. In order to provide a clearer and consistent understanding of the specification and claims, including the scope to be given such terms, the following definitions are provided.

In general, an ‘epitope’ is defined as a linear array of 3-20 amino acids aligned along the surface of a protein. In a linear epitope, the amino acids are joined sequentially and follow the primary structure of the protein. In a conformational epitope, residues are not joined sequentially, but lie linearly along the surface due to the conformation (folding) of the protein. With respect to conformational epitopes, the length of the epitope-defining sequence can be subject to wide variations. The portions of the primary structure of the antigen between the residues defining the epitope may not be critical to the structure of the conformational epitope. For example, deletion or substitution of these intervening sequences may not affect the conformational epitope provided sequences critical to epitope conformation are maintained (e.g. cysteines involved in disulfide bonding, glycosylation sites, etc.). A conformational epitope may also be formed by 2 or more essential regions of subunits of a homo-oligomer or hetero-oligomer. As used herein, ‘epitope’ or ‘antigenic determinant’ means an amino acid sequence that is immunoreactive. As used herein, an epitope of a designated polypeptide denotes epitopes with the same amino acid sequence as the epitope in the designated polypeptide, and immunologic equivalents thereof. Such equivalents also include strain, subtype (=genotype), or type (group)-specific variants, e.g. of the currently known sequences or strains belonging to Plasmodium such as 3D7, FVO, Camp, NF54, and T9/96, or any other known or newly defined Plasmodium strain.

The term ‘solid phase’ intends a solid body to which the individual P. falciparum antigen is bound covalently or by noncovalent means such as hydrophobic, ionic, or van der Waals association.

The term ‘biological sample’ intends a fluid or tissue of a mammalian individual (e.g. an anthropoid, a human), reptilian, avian, or any other zoo or farm animal that commonly contains antibodies produced by the individual, more particularly antibodies against malaria. The fluid or tissue may also contain P. falciparum antigen. Such components are known in the art and include, without limitation, blood, plasma, serum, urine, spinal fluid, lymph fluid, secretions of the respiratory, intestinal or genitourinary tracts, tears, saliva, milk, white blood cells and myelomas. Body components include biological liquids. The term ‘biological fluid’ refers to a fluid obtained from an organism.

The term ‘immunologically reactive’ means that the antigen in question will react specifically with anti-AMA-1 antibodies, present in vitro or in a body component from a malaria infected individual.

The term ‘immune complex’ intends the combination formed when an antibody binds to an epitope on an antigen.

The term ‘recombinant’ used within the context of the present invention refers to the fact that the proteins of the present invention are produced by recombinant expression methods be it in prokaryotes, or lower or higher eukaryotes as discussed in detail below.

The term ‘polypeptide’ refers to a polymer of amino acids and does not refer to a specific length of the product; thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogues of an amino acid (including, for example, unnatural amino acids, PNA, etc.), polypeptides with substituted linkages, as well as other modifications known in the art, both naturally occurring and non-naturally occurring.

The term ‘recombinant polynucleotide or nucleic acid’ intends a polynucleotide or nucleic acid of genomic, cDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature.

The term ‘recombinant host cells’, ‘host cells’, ‘cells’, ‘cell lines’, ‘cell cultures’, and other such terms denoting microorganisms or higher eukaryotic cell lines cultured as unicellular entities refer to cells which can be or have been, used as recipients for a recombinant vector or other transfer polynucleotide, and include the progeny of the original cell which has been transfected. It is understood that the progeny of a single parental cell may not necessarily be completely identical in morphology or in genomic or total DNA complement as the original parent, due to natural, accidental, or deliberate mutation.

The term ‘immunogenic’ refers to the ability of a substance to cause a humoral and/or cellular response, whether alone or when linked to a carrier, in the presence or absence of an adjuvant. ‘Neutralization ’ refers to an immune response that blocks the infectivity, either partially or fully, of an infectious agent. A ‘vaccine ’ is an immunogenic composition capable of eliciting protection against malaria, whether partial or complete. A vaccine may also be useful for treatment of an infected individual, in which case it is called a therapeutic vaccine.

The term “polyclonal” refers to a mixture of antibodies directed against several epitopes on a molecule. The term, monoclonal, refers to a antibodies against a specific epitope on a molecule. Fab fragments refers to the monomeric antibody binding units of IgG antibodies generated after papain treatment.

This application describes the first observations of the effects of invasion-inhibitory anti-AMA-1 antibodies on parasite AMA-1 processing and redistribution. The present invention is based on our discovery that antibodies against AMA-1 that inhibit invasion of the parasite against in an in vitro invasion assay act by inhibiting the processing of AMA-1 because the processing-inhibitory potential of a serum sample correlates with its ability to inhibit invasion in vitro. Assay based on detecting AMA-1 processing inhibition could be used to test immunity provided after administration of an AMA-1-based vaccine or immune therapy.

The method comprises incubating a sample suspected of containing anti-AMA-1 invasion-inhibitory antibodies or processing inhibitory antibodies with P. falciparum synchronized schizonts, preferably, mid-stage schizonts (about 8 nuclei) and >90% pure (1×10⁷ per ml), between 90-95% pure. Following the rupture of these schizonts, PfAMA-1 processing products and intermediates can be detected by resolving and quantifying immunoprecipitated antigens. We have observed that an increase in the amount of PfAMA-1₆₆, PfAMA-1₅₂ and PfAMA-1₁₀ indicates that the antibodies are invasion inhibitory. One of the methods to calculate the processing inhibitory potential of an immune sera is by calculating the ratio of PfAMA-1₁₀/(PfAMA-1₁₀+PfAMA-1₂₀)×100 and comparing it to the one obtained for the positive and negative standards that are assayed alongside as described in the Examples below.

In another aspect of the invention, the shed AMA-1 may be used to quantitate the ability of a serum sample to inhibit AMA-1 processing and shedding. The method comprises incubating a sample suspected of containing anti-AMA-1 invasion-inhibitory antibodies or processing inhibitory antibodies with P. falciparum synchronized schizonts, and collecting the supernatant for analysis on western blots. The presence the of 44+48 kDa forms of AMA-1 equivalent to the negative controls would suggest that the antibodies are not processing inhibitory and possibly not invasion inhibitory either. However, if the amount of the 44+48 forms in the test serum is less than the controls, it would indicate that the serum sample is processing and invasion inhibitory.

We have also found that polyclonal antibodies against AMA-1 can inhibit the redistribution of AMA-1 on the merozoite surface. To detect this schizonts can be allowed to rupture in the presence of the antibodies to be tested in various dilutions. Following rupture, the merozoites can be smeared on a slide and immuno-stain for the localization of AMA-1 using antibodies against AMA-1 that can be labeled to observe whether or not redistribution is inhibited. If AMA-1 redistribution is found to be inhibited, as observed under a fluorescence microscope, this would indicate that the serum sample inhibited the redistribution of AMA-1 and hence it may be inhibitory in an invasion assay. We believe that this information may be potentially useful in developing other in vitro correlates of AMA-1 immunity.

To this date, the sheddase responsible for proteolytically cleaving AMA-1 on the merozoite has not been identified. However, it is envisioned that an assay can be developed using recombinant forms of AMA-1 sheddases for detecting the presence of processing inhibitory antibodies in a sample. Such an assay would eliminate the need for parasites. The sheddase can be used in combination with affinity purified AMA-1 protein as a substrate, and can be used to measure the in vitro processing inhibition caused by test antibodies. Kits for performing the assay would include a recombinant AMA-1, a recombinant sheddase, control positive and negative antibody standards, and the required buffers and components to perform the assay.

The present invention also contemplates a kit for performing the diagnostic assay above, said kit comprising:
(i) buffers or components necessary for parasite growth and analysis of parasitic growth stage;
(ii) sera or a combination of antibodies known to inhibit processing to serve as a positive control and sera or a combination of antibodies known to not inhibit AMA-1 processing to serve as a negative control, and buffers or components necessary for dissolving or diluting these factors.

(iii) buffers and components necessary for analyzing the immune complexes;
(iv) means for detecting the immune complexes formed, such as labeled secondary antibodies
(v) possibly also including an automated scanning and interpretation device for analyzing and quantifying the immune complexes.

The present invention also relates to a method for in vitro diagnosis of malaria antibodies present in a biological sample, comprising at least the following steps

The immunoassay methods according to the present invention utilize domains that maintain linear and conformational epitopes recognized by antibodies in the sera from vaccinated individuals or individuals infected with a malaria parasite. The AMA-1 processing products or antigens of the present invention may be employed in virtually any assay format that employs a known antigen to detect antibodies. A common feature of all of these assays is that the antigen is contacted with the body component suspected of containing malaria antibodies under conditions that permit the antigen to bind to any such antibody present in the component. Such conditions will typically be physiologic temperature, pH and ionic strength using an excess of antigen. The incubation of the antigen with the specimen is followed by detection of immune complexes comprised of the antigen and antibody.

Design of the immunoassays is subject to a great deal of variation, and many formats are known in the art. Protocols may, for example, use solid supports, or immunoprecipitation. Most assays involve the use of labeled antibody or polypeptide; the labels may be, for example, enzymatic, fluorescent, chemiluminescent, radioactive, or dye molecules. Assays which amplify the signals from the immune complex are also known; examples of which are assays which utilize biotin and avidin or streptavidin, and enzyme-labeled and mediated immunoassays, such as ELISA assays.

The immunoassay may be, without limitation, in a heterogeneous or in a homogeneous format, and of a standard or competitive type. In a heterogeneous format, the polypeptide is typically bound to a solid matrix or support to facilitate separation of the sample from the polypeptide after incubation. Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride (known as Immunolon.™.), diazotized paper, nylon membranes, activated beads, and Protein A beads. For example, Dynatech Immunolon.™.1 or Immunlon.™. 2 micrometer plates or 0.25 inch polystyrene beads (Precision Plastic Ball) can be used in the heterogeneous format. The solid support containing the antigenic polypeptides is typically washed after separating it from the test sample, and prior to detection of bound antibodies. Both standard and competitive formats are known in the art.

In a homogeneous format, the test sample is incubated with the combination of antigens in solution. For example, it may be under conditions that will precipitate any antigen-antibody complexes which are formed. Both standard and competitive formats for these assays are known in the art.

In a standard format, the amount of malaria antibodies in the antibody-antigen complexes is directly monitored. This may be accomplished by determining whether labeled anti-xenogeneic (e.g. anti-human) antibodies which recognize an epitope on anti-malaria antibodies will bind due to complex formation. In a competitive format, the amount of malaria antibodies in the sample is deduced by monitoring the competitive effect on the binding of a known amount of labeled antibody (or other competing ligand) in the complex.

Complexes formed comprising anti-malaria antibody (or in the case of competitive assays, the amount of competing antibody) are detected by any of a number of known techniques, depending on the format. For example, unlabeled malaria antibodies in the complex may be detected using a conjugate of anti-xenogeneic Ig complexed with a label (e.g. an enzyme label).

In an immunoprecipitation or agglutination assay format the reaction between the malaria antigens and the antibody forms a network that precipitates from the solution or suspension and forms a visible layer or film of precipitate. If no anti-malaria antibody is present in the test specimen, no visible precipitate is formed.

There currently exist three specific types of particle agglutination (PA) assays. These assays are used for the detection of antibodies to various antigens when coated to a support. One type of this assay is the hemagglutination assay using red blood cells (RBCs) that are sensitized by passively adsorbing antigen (or antibody) to the RBC. The addition of specific antigen antibodies present in the body component, if any, causes the RBCs coated with the purified antigen to agglutinate.

To eliminate potential non-specific reactions in the hemagglutination assay, two artificial carriers may be used instead of RBC in the PA. The most common of these are latex particles. However, gelatin particles may also be used. The assays utilizing either of these carriers are based on passive agglutination of the particles coated with purified antigens.

The AMA-1 proteins, polypeptides, or antigens of the present invention will typically be packaged in the form of a kit for use in these immunoassays. The kit will normally contain in separate containers the AMA-1 antigens or processing products, control antibody formulations (positive and/or negative), labeled antibody when the assay format requires the same and signal generating reagents (e.g. enzyme substrate) if the label does not generate a signal directly. The antigens may be already bound to a solid matrix or separate with reagents for binding it to the matrix. Instructions (e.g. written, tape, CD-ROM, etc.) for carrying out the assay usually will be included in the kit.

Immunoassays that utilize the AMA-1 processing antigens are useful in screening blood for the preparation of a supply from which potentially infective malaria parasite is lacking. The method for the preparation of the blood supply comprises the following steps. Reacting a body component, preferably blood or a blood component, from the individual donating blood with AMA-1 proteins of the present invention to allow an immunological reaction between malaria antibodies, if any, and the AMA-1 antigen. Detecting whether anti-malaria antibody-AMA-1 antigen complexes are formed as a result of the reacting. Blood contributed to the blood supply is from donors that do not exhibit antibodies to the native AMA-1 antigens.

The present invention also relates to an antibody against PfAMA-1 proteolytic processing product such as PfAMA-1₅₂, PfAMA-1₄₄₊₄₈, PfAMA-1₂₀ and PfAMA-1₁₀ The antibody can be screened from a variable chain library in plasmids or phages or from a population of human B-cells by means of a process known in the art, with said antibody being reactive with any of the polypeptides or peptides as defined above, and with said antibody being preferably a monoclonal antibody.

The PfAMA-1 proteolytic processing product specific monoclonal antibodies of the invention can be produced by any hybridoma liable to be formed according to classical methods from splenic or lymph node cells of an animal, particularly from a mouse or rat, immunized against the Plasmodium polypeptides or peptides according to the invention, as defined above on the one hand, and of cells of a myeloma cell line on the other hand, and to be selected by the ability of the hybridoma to produce the monoclonal antibodies recognizing the polypeptides which has been initially used for the immunization of the animals.

The antibodies involved in the invention can be labeled by an appropriate label of the enzymatic, fluorescent, or radioactive type.

The monoclonal antibodies according to this preferred embodiment of the invention may be humanized versions of mouse monoclonal antibodies made by means of recombinant DNA technology, departing from parts of mouse and/or human genomic DNA sequences coding for H and L chains from cDNA or genomic clones coding for H and L chains.

Alternatively the monoclonal antibodies according to this preferred embodiment of the invention may be human monoclonal antibodies. These antibodies according to the present embodiment of the invention can also be derived from human peripheral blood lymphocytes of patients infected with malaria, or vaccinated against malaria. Such human monoclonal antibodies are prepared, for instance, by means of human peripheral blood lymphocytes (PBL) repopulation of severe combined immune deficiency (SCID) mice, or by means of transgenic mice in which human immunoglobulin genes have been used to replace the mouse genes.

The invention also relates to the use of the proteins or peptides of the invention, for the selection of recombinant antibodies by the process of repertoire cloning.

Antibodies directed to peptides or single or specific proteins derived from a certain strain may be used as a medicament, more particularly for incorporation into an immunoassay for the detection of Plasmodium strains for detecting the presence of PfAMA-1 antigens, or antigens containing PfAMA-1 epitopes, for prognosing/monitoring of malaria disease, or as therapeutic agents.

Alternatively, the present invention also relates to the use of any of the above-specified PfAMA-1 processing products monoclonal antibodies in an assay for proteolytic processing of AMA-1, for the preparation of an immunoassay kit for detecting the presence of AMA-1 processing antigen(s) or antigens containing AMA-1 epitopes in a biological samples, for the preparation of a kit for prognosing/monitoring of malaria disease or for the preparation of a malaria medicament.

Monoclonal antibodies according to the present invention are suitable both as therapeutic and prophylactic agents for treating or preventing malaria infection in susceptible malaria-infected subjects.

In general, this will comprise administering a therapeutically or prophylactically effective amount of one or more monoclonal antibodies of the present invention to a susceptible subject or one exhibiting malaria infection. Any active form of the antibody can be administered, including Fab and F(ab′)₂ fragments. Antibodies of the present invention can be produced in any system, including insect cells, baculovirus expression systems, chickens, rabbits, goats, cows, or plants such as tomato, potato, banana or strawberry. Methods for the production of antibodies in these systems are known to a person with ordinary skill in the art. Preferably, the antibodies used are compatible with the recipient species such that the immune response to the MAbs does not result in clearance of the MAbs before parasite can be controlled, and the induced immune response to the MAbs in the subject does not induce “serum sickness” in the subject. Preferably, the MAbs administered exhibit some secondary functions such as binding to Fc receptors of the subject.

Treatment of individuals having malaria infection may comprise the administration of a therapeutically effective amount of one or more AMA-1 processing product antibodies of the present invention. The antibodies can be provided in a kit as described below. The antibodies can be used or administered as a mixture, for example in equal amounts, or individually, provided in sequence, or administered all at once. In providing a patient with antibodies, or fragments thereof, capable of binding to AMA-1 processing products, or an antibody capable of protecting against malaria in a recipient patient, the dosage of administered agent will vary depending upon such factors as the patient's age, weight, height, sex, general medical condition, previous medical history, etc.

In general, it is desirable to provide the recipient with a dosage of antibody which is in the range of from about 1 pg/kg-100 pg/kg, 100 pg/kg-500 pg/kg, 500 pg/kg-1 ng/kg, 1 ng/kg-100 ng/kg, 100 ng/kg-500 ng/kg, 500 ng/kg-1 ug/kg, 1 ug/kg-100 ug/kg, 100 ug/kg-500 ug/kg, 500 ug/kg-1 mg/kg, 1 mg/kg-50 mg/kg, 50 mg/kg-100 mg/kg, 100 mg/kg-500 mg/kg, 500 mg/kg-1 g/kg, 1 g/kg-5 g/kg, 5 g/kg-10 g/kg (body weight of recipient), although a lower or higher dosage may be administered.

In a similar approach, another prophylactic use of the monoclonal antibodies of the present invention is the active immunization of a patient using an anti-idiotypic antibody raised against one of the present monoclonal antibodies. Immunization with an anti-idiotype which mimics the structure of the epitope could elicit an active anti-AMA-1 response (Linthicum, D. S. and Farid, N. R., Anti-Idiotypes, Receptors, and Molecular Mimicry (1988), pp 1-5 and 285-300).

Likewise, active immunization can be induced by administering one or more antigenic and/or immunogenic epitopes as a component of a subunit vaccine. Vaccination could be performed orally or parenterally in amounts sufficient to enable the recipient to generate protective antibodies or immunoreactive T-cells against AMA-1 in a manner that has either prophylactical or therapeutical value. The host can be actively immunized with the antigenic/immunogenic peptide in pure form, a fragment of the peptide, or a modified form of the peptide. One or more amino acids, not corresponding to the original protein sequence can be added to the amino or carboxyl terminus of the original peptide, or truncated form of peptide. Such extra amino acids are useful for coupling the peptide to another peptide, to a large carrier protein, or to a support. Amino acids that are useful for these purposes include: tyrosine, lysine, glutamic acid, aspartic acid, cyteine and derivatives thereof. Alternative protein modification techniques may be used e.g., NH₂-acetylation or COOH-terminal amidation, to provide additional means for coupling or fusing the peptide to another protein or peptide molecule or to a support.

The antibodies capable of protecting against malaria are intended to be provided to recipient subjects in an amount sufficient to effect a reduction in the malaria infection symptoms. An amount is said to be sufficient to “effect” the reduction of infection symptoms if the dosage, route of administration, etc. of the agent are sufficient to influence such a response. Responses to antibody administration can be measured by analysis of subject's vital signs.

All documents cited herein supra and infra are hereby incorporated by reference thereto.

Administration of the compounds disclosed herein may be carried out by any suitable means, including parenteral injection (such as intraperitoneal, subcutaneous, or intramuscular injection), orally, or by topical application of the antibodies (typically carried in a pharmaceutical formulation) to an airway surface. Topical application of the antibodies to an airway surface can be carried out by intranasal administration (e.g., by use of dropper, swab, or inhaler which deposits a pharmaceutical formulation intranasally). Topical application of the antibodies to an airway surface can also be carried out by inhalation administration, such as by creating respirable particles of a pharmaceutical formulation (including both solid particles and liquid particles) containing the antibodies as an aerosol suspension, and then causing the subject to inhale the respirable particles. Methods and apparatus for administering respirable particles of pharmaceutical formulations are well known, and any conventional technique can be employed. Oral administration may be in the form of an ingestable liquid or solid formulation.

The treatment may be given in a single dose schedule, or preferably a multiple dose schedule in which a primary course of treatment may be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease.

The contents of all cited references (including literature references, issued patents, published patent applications, and co-pending patent applications) cited throughout this application are hereby expressly incorporated by reference.

Other features of the invention will become apparent in the course of the following descriptions of exemplary embodiments which are given for illustration of the invention and are not intended to be limiting thereof.

The following Materials and Methods were used in the Examples below.

Antibodies. Rabbit antibodies were raised against recombinant AMA-1 (449 amino acids of P. falciparum 3D7 clone or FVO strain, or a mixture of the two proteins, residue # 83Gly-531Glu) (Dutta et al., 2002, Infect. Immun. 70, 3101-3110). Pooled or individual serum samples were used in the study. A pool of pre-immune and adjuvant control rabbit sera served as control. IgG's were purified using 1 ml Protein G column (Amersham). Fab fragments were prepared from IgG by papain digestion (Antibodies, A Laboratory Manual, 1988, eds. Harlow, E. and Lane, D. (Cold Spring Harbor Laboratory) pp 626-629) using ImmunoPure Fab kit (Pierce). Purity of the Fab fragments preparation was confirmed by SDS-PAGE.

Polyclonal IgG against recombinant AMA-1 was labeled with biotin using the EZ-link Biotinylation kit (Pierce). Monoclonal antibody (mAb) 4G2dc1 reacts with a conformational epitope on the ectodomain of AMA-1 (Kocken et al., 1998, supra) and mAb 5.2 recognizes the MSP19 of P. falciparum (Chang et al., 1988, Exp. Parasitol. 67, 1-11).

Rat monoclonal antibodies 4G2dc1 and 28G2dc1 (directed against the extreme C-terminus sequence) and mAb 58F8dc1 (directed against an N-terminal peptide of PfAMA-1) were kindly provided by Dr. Alan Thomas, Biomedical Primate Research Center, Rijswijk, The Netherlands.

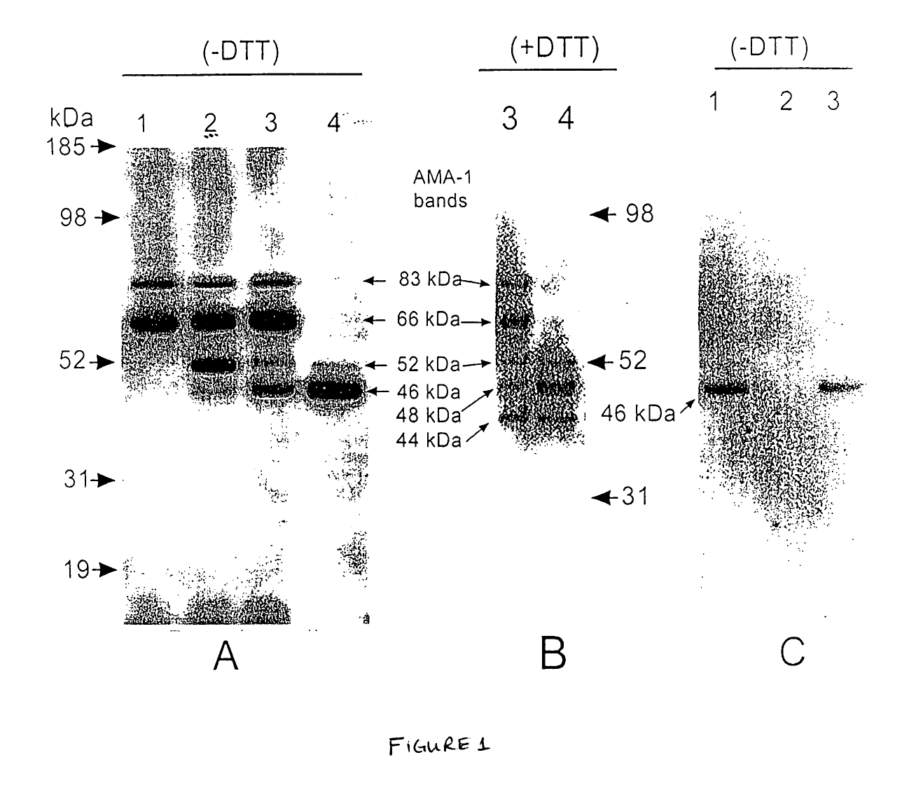

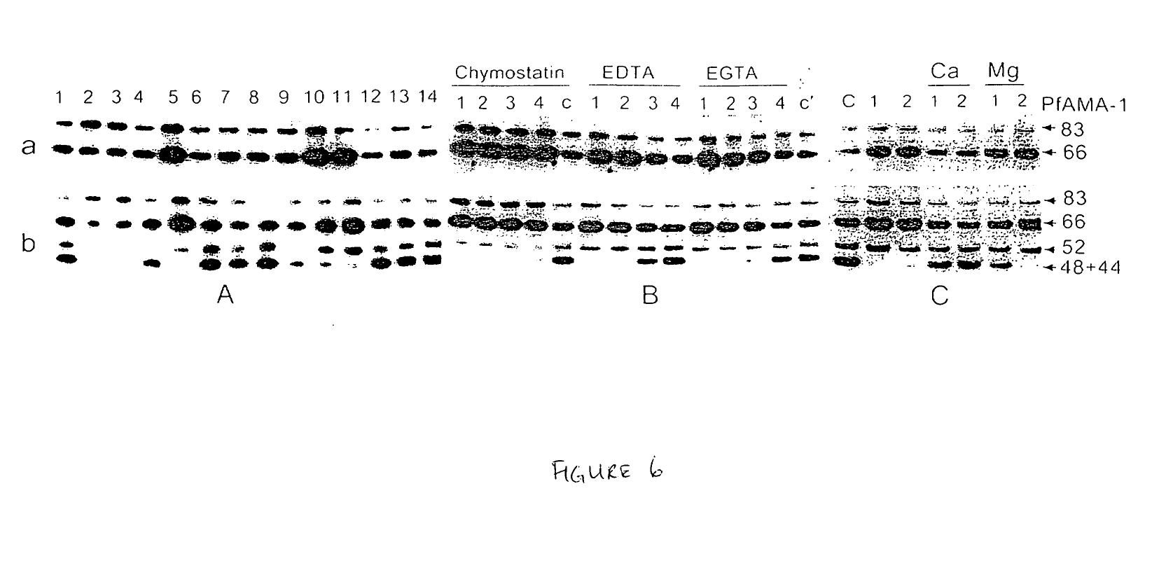

AMA-1 processing assay, or processing inhibition assay (PIA). P. falciparum clone 3D7 cultures were prepared as described previously (Haynes and Moch, 2002, Methods Mol. Med. 72, 489-497), culture media included 10% heat inactivated normal human serum in bicarbonate-containing RPMI 1640 containing final 0.42 mM Ca⁺² and 0.40 mM Mg⁺². Fifteen microliters of heat inactivated rabbit serum (dialyzed against RPMI 1640 or used directly after heat inactivation) or purified IgG or Fab fragments was mixed with 135 μl of synchronized (Haynes and Moch, 2002, supra), Percoll-alanine purified (Kanaani and Ginsburg, 1989, J. Biol. Chem. 264, 3194-3199), mid-stage (˜8 nuclei), >90% pure schizonts (1×10⁷ per ml) in a 48 well culture plate. PBS was used as a diluent if necessary. The plate was placed in a plastic bag, gassed with 5% O₂-5% CO₂, heatsealed, and incubated at 37° C. for ˜6 h (Haynes et al., 2002, Methods Mol. Med. 72, 535-554). Aliquots from a control culture flask were taken to monitor the percent rupture of schizonts by hemocytometer. After ˜90% schizonts had ruptured, the contents of each well were transferred to a microfuge tube and centrifuged at 10,000× g for 5 min. The supernatant was aspirated and the resulting parasite pellet was washed with 0.5 ml chilled PBS and centrifuged as before. The supernatant was discarded and 150 μl of 1× NuPAGE sample buffer (Invitrogen) was added to the tubes, samples were frozen at −30° C. until analyzed. Soluble forms of AMA-1 were immuno-precipitated from the processing assay culture supernatants or from culture supernatant of routinely maintained parasites using MagnaBind Goat anti-Rabbit IgG coated Magnetic Beads (Pierce). Two types of western blots have been used to detect AMA-1 fragments. First set of westerns in FIGS. 1 to 6 have been developed using biotinylated polyclonal IgG against recombinant AMA-1. A second set of western blots, FIG. 6 onwards, has been developed using two biotinylated primary antibodies, mAb 28G2dc1 (1:1000) and polyclonal anti-AMA-1 IgG (1:1000) used simultaneously in order to immunostain all the AMA-1 specific fragments on the blot.

Protease inhibitors. Protease inhibitors were tested for their effect on AMA-1 processing. All inhibitors were from Sigma Chemicals. PMSF, pepstatin and TPCK were dissolved in 100% ethanol. Antipain, leupeptin, and 1,10-phenanthroline stocks were made in water. EDTA and EGTA stocks were made in PBS (pH adjusted to 7.2), TLCK was prepared in 1 mM HCl, E64 and chymostatin were prepared in DMSO. All stocks were at 100× concentration. 1.5 μl of the protease inhibitor or its respective solvent control was added to 15 μl PBS and then 135 μl of (1×10⁷/ml) Percoll purified mid-stage schizonts were added. The processing assay was carried out as described above. Parallel assay with protease inhibitors was carried out in two similarly prepared plates to which RBC were also added to a final 4% hematocrit. Parasites in the first plate were allowed to invade in suspension at 37° C. and thin smears were prepared after the rupture cycle for giemsa staining and examination for the presence of Protease inhibitor Clusters of Merozoites (PCM) (Lyon and Haynes, 1986, J. Immunol. 136, 2245-2251). In the second plate parasites (135 μl of 5×10⁶/ml schizonts+RBC at 4% hematocrit) were incubated overnight and ring forms were quantitated by flow cytometry as a measure of parasite invasion (Haynes et al., 2002, supra).

PAGE and Western blotting. Samples were briefly sonicated with a microtip sonicator, heated at 80° C. for 2 min, spun down and 16 μl was applied per well to a precast 4-12% gradient polyacrylamide gel (NuPAGE Bis-Tris; Invitrogen). Samples were run under non-reduced conditions unless specified. DTT at 50 mM was added to samples resolved under reduced conditions. Proteins from the gel were electrophoretically transferred to nitrocellulose membrane and blocked with 5% BSA in PBS containing 0.05% Tween-20 overnight at 4° C. AMA-1 specific bands were immunostained by incubating the blot with primary biotin-labeled polyclonal antibody for 2 h. Reducing western blot were immunostained with biotin labeled polyclonal IgG against reduced and alkylated recombinant AMA-1. Following washing with PBS-Tween, HRP conjugated NeutrAvidin™ (Pierce) at 1:15,000 or 1:10,000 dilution was added for 1 h, the blot was washed and developed with SuperSignal™ West Pico Chemiluminescent substrate (Pierce) followed by X-ray film exposure (Kodak BioMax). Developed X-ray films were scanned and band intensity was calculated using ImageQuant 5.1 software (Molecular Dynamics).

Western blots developed with a HRP Chemiluminescent substrate (WestPico, Pierce) and exposed to X-ray films (Kodak BioMax). Developed blots were scanned and densitometric analysis was performed on scanned images. ImageQuest 5.1 (Molecular Dynamics) software was used for the analysis and pixel density was calculated by placing rectangles of equal area on individual bands. Data analysis was performed on Microsoft Excel software.

When using monoclonal antibodies, the pellet was resuspended in 100 ul sample buffer, briefly sonicated and run on a 4-20% SDS-PAGE, under non-reduced conditions (NUPage, Invitrogen). The proteins were electrophoretically transferred to a nitrocellulose membrane and the blot was blocked with 5% BSA in PBS for 2 hours. Primary antibody constituted a mixture of biotinylated mAb 28G2dc1 and polyclonal anti-AMA-1 IgG (2 mg/ml each, 1:1000 dilution). Secondary antibody was HRP-conjugated Neutravidin (Pierce, 1:10,000 dilution). All incubations were for 2 hr at room temperature and blot was washed with PBS containing 0.05% Tween-20. Western blot was developed using SuperSignal West Pico Chemiluminescent substrate (Pierce), followed by x-ray film exposure (Kodak Biomax).

AMA-1 localization using Indirect Immunofluorescence assay (IFA). Midstage schizonts were incubated with the antibodies in the same format as described in the processing assay. At ˜70% schizont rupture samples were chilled and a protease inhibitor cocktail (Cat # 554779, Pharmingen) was added to each sample. In order to have similar concentration of the post-immune sera in the test and control wells, post-immune anti-AMA-1 sera (1:10) was added to wells corresponding to pre-immune or negative serum control and all samples were incubated on ice for another 1.5 h to allow the newly added antibodies to bind. This was followed by centrifugation at 2000× g for 4 min and the resulting pellet was washed with growth medium containing 10% human serum and protease inhibitor cocktail. The final pellet was suspended in 20 μl wash buffer and 1 μl was spotted for IFA. Slides were fixed with acetone for 1 min and blocked with 10 mg/ml BSA in PBS for 30 min. Primary antibody was anti-MSP-1 mouse mAb 5.2 (1:1000 of ascitic fluid), secondary antibodies were anti-rabbit FITC conjugated antibody (1:300) and anti-mouse Phycoerythrin conjugate (1:1000) (All from Southern Biotechnology Associates). Both incubations were for 1 h. The slides were washed with PBS and mounted with Fluoromount G. Microscopy was done under UV with FITC filter for AMA-1 and dual cube FITC+PE filter for co-localizing AMA-1 and MSP-1. In IFA for protease inhibitor treated parasites, 1:1000 dilution of polyclonal anti-AMA-1 rabbit sera was added concurrently with mAb 5.2.

ELISA and Growth Inhibition Assay (GIA). ELISA and static GIA were done as described previously (Dutta et al., 2002, supra; Haynes et al., 2002, supra). Secondary antibodies anti rabbit H+L and anti Rat H+L were obtained from Southern Biotech.

### EXAMPLE 1

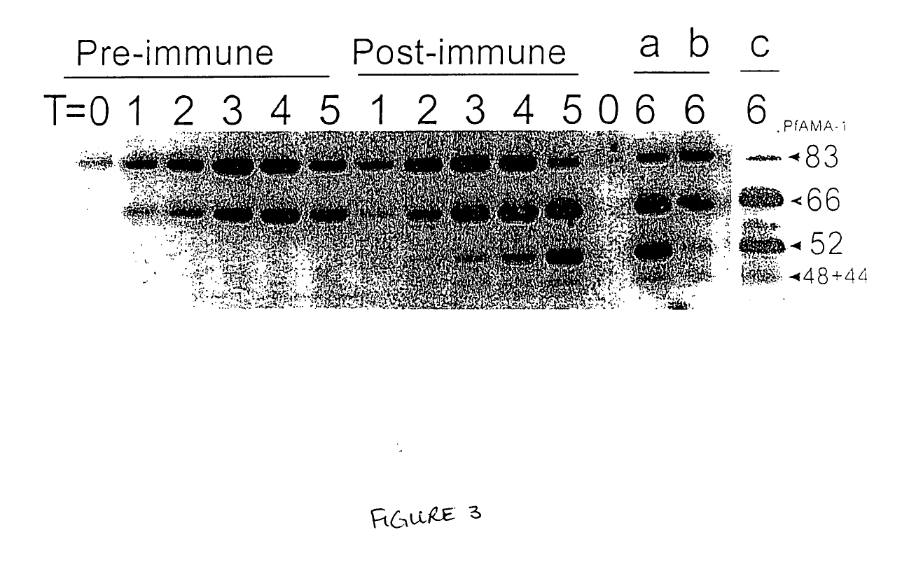

Processing of AMA-1 in the presence of anti-AMA-1. A processing assay was performed with pre-immune (1:10) (non-inhibitory by GIA), immune 1:10 (>85% inhibition by GIA) and immune 1:2500 (non-inhibitory by GIA) serum pools. The resulting parasite pellets were analyzed by western blotting. Immunoprecipitated soluble AMA-1 fragments from culture supernatant of routinely maintained parasites, using polyclonal anti-AMA-1, was also analyzed on the same gel. As expected, two AMA-1 specific bands were detected under non-reduced conditions in merozoites released in the presence of pre-immune serum (FIG. 1A, lane 1). These bands migrated at 73 and 62 kDa (under our electrophoretic conditions); and as evidenced by reactivity to mAb 4G2dc1 (FIG. 3 lane-c), correspond to the 83 and 66 kDa forms respectively, of PfAMA-1 fragments described in the literature. In contrast, merozoites released in the presence of anti-AMA-1 sera showed two additional bands at 52 and 46 kDa respectively (FIG. 1A, lanes 2, 3). The 52:46 band intensity ratio was higher at 1:10 dilution (FIG. 1A, lane 2) as compared to 1:2500 (FIG. 1A, lane 3). The 52 and 46 kDa bands had similar mobility to the soluble forms of PfAMA-1 observed in the positive immuno-precipitation control (FIG. 1A, lane 4). A recent publication (Howell et al., 2003, J. Biol. Che. 278, 23890-23898) reported AMA-1 fragments immunoprecipitated from culture supernatant migrate at 52 and 46 kDa under non-reduced conditions, and that the 46 kDa band constitutes co-migrating 48 and 44 kDa soluble forms. The 46 kDa band observed in our processing assay also resolved into a 48 and 44 kDa band under reducing conditions (FIG. 1B, lanes 3, 4). Hence, it was concluded that the 52 and 46 kDa bands observed on our processing assays under non reduced conditions represent the 52 and 46 kDa bands observed by others (Howell et al., 2003, supra) and for the purpose of maintaining continuity with published data we will refer to the observed 73, 62, 52 and 46 kDa bands as PfAMA-1_{83, 66, 52 and 48+44}, respectively. Our data demonstrate that invasion inhibitory anti-AMA-1 can trap an intermediate form PfAMA-1₅₂ along with the soluble forms PfAMA-1₄₈₊₄₄ on the merozoite surface.

In the same experiment the shed fragments of AMA-1 were immuno-precipitated from culture supernatant and analyzed under non-reducing conditions. The 46 kDa band was detected in the supernatant from parasites incubated with pre-immune (FIG. 1C, lane 1) and 1:2500 immune sera (FIG. 1C, lane 3), while it was absent in the supernatant from parasites incubated with 1:10 immune serum (FIG. 1C, lane 2). The results indicate that at inhibitory concentration, antibodies to AMA-1 inhibit the formation and shedding of PfAMA-1₄₈₊₄₄ from merozoites.

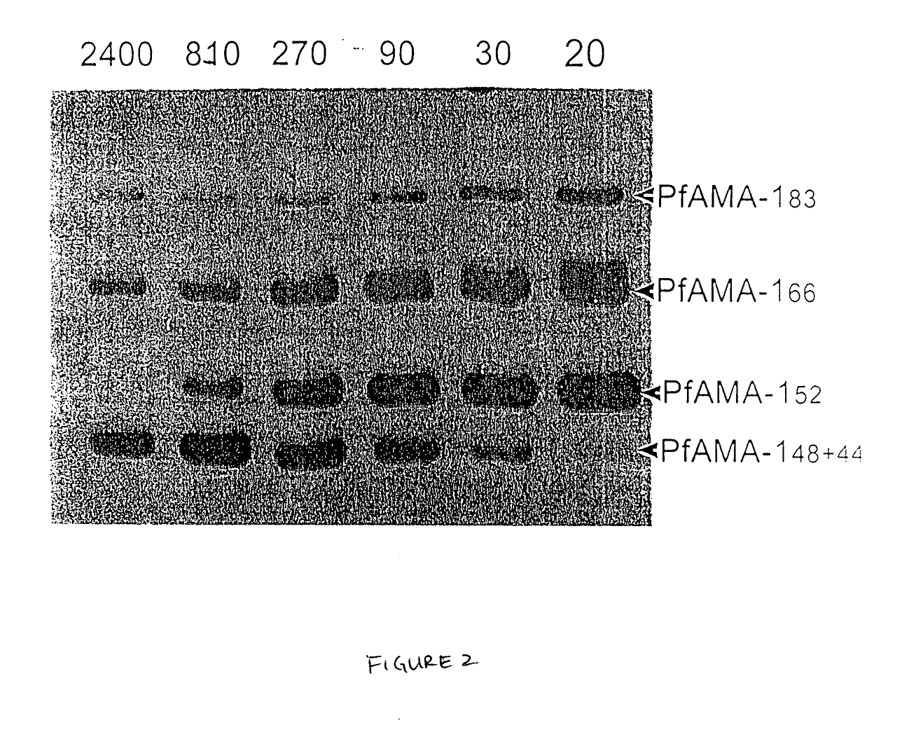

An individual inhibitory rabbit serum was tested in a processing assay at 20, 30, 90, 270, 810 and 2400 fold dilution respectively (FIG. 2). Four distinct effects of antibodies on AMA-1 processing were observed. First, PfAMA-1₅₂ and PfAMA-1₄₈₊₄₄ were trapped on the merozoites. Second, the formation of PfAMA-1₄₈₊₄₄ appeared to be inhibited by antibodies and the ratio of PfAMA-1₅₂ to PfAMA-1₄₈₊₄₄ was higher at higher concentrations of the inhibitory sera. Third, high concentration of inhibitory anti-AMA-1 sera led to substantial accumulation of the PfAMA-1₆₆ (FIG. 2, compare 1:20 and 1:270 dilutions), suggesting processing inhibition of PfAMA-1₆₆.

### EXAMPLE 2

Kinetics and specificity of the processing assay. AMA-1 is synthesized and processed during schizont development and rupture (Narum and Thomas, 1994, supra; Crewther et al., 1990, supra; Healer et al, 2002, supra; Kocken et al., 1998, supra; Howell et al., 2001, supra; Howell et al., 2003, supra). In order to determine if the processing assay can detect the synthesis and processing of AMA-1, schizonts were allowed to rupture in the presence of inhibitory immune serum pool at a 1:10 dilution (FIG. 3). A pool of control pre-immune serum was also incubated at identical concentration. Schizont rupture was monitored by hemocytometer counts. No difference was observed in the rupture kinetics of schizonts in pre- or post-immune sera. Starting at T0 (corresponding to 0% rupture) sample sets were drawn at T1 (2.25 h; 30% rupture), T2 (3 h; 40%), T3 (4.25 h; 60%), T4 (5.5 h; 76%), T5 (6.5 h; 87%) and analyzed by Western blotting under non-reduced conditions. In the pre-immune control lanes, PfAMA-1₈₃ and PfAMA-1₆₆ were detected. While in the lanes corresponding to immune serum, additional PfAMA-1₅₂ and PfAMA-1₄₈₊₄₄ bands were also seen. The relative intensity of PfAMA-1₅₂ and PfAMA-1₄₈₊₄₄ remained unchanged over time. In order to rule out immuno-precipitation of AMA-1 from the culture supernatant, immune serum was added (at T5) to one of the pre-immune wells and incubation continued at 37° C. for an additional 30 min (T6) PfAMA-1₅₂ and PfAMA-1₄₈₊₄₄ bands in this control lane were much weaker (lane-b) compared to when immune sera was present from the beginning (lane-a), suggesting that the assay detects proteolytic products of cross-linked membrane bound AMA-1 molecules. Reactivity to mAb 4G2dc1 was further used to confirm the identity of the observed bands (lane-c).

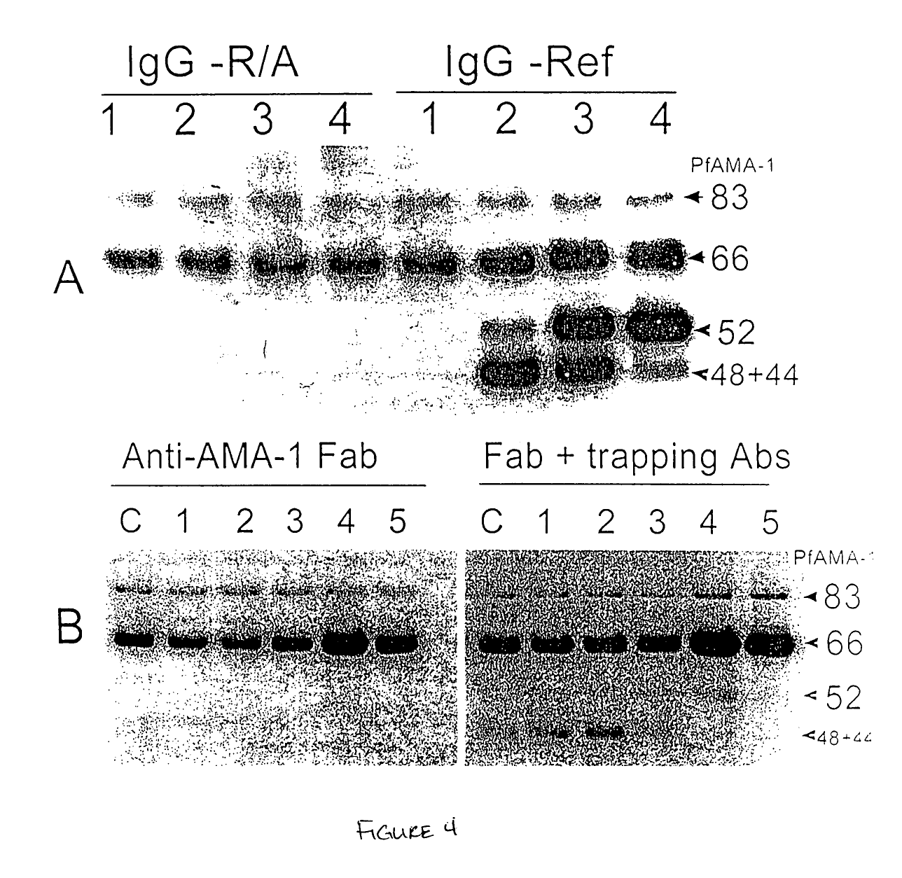

IgG prepared from inhibitory serum pool of rabbits immunized with refolded recombinant AMA-1 (Dutta et al., 2002, supra) and IgG isolated from non-inhibitory serum of rabbits immunized with reduced and alkylated recombinant AMA-1, were tested in a processing assay. Table 1 shows ELISA titers and GIA activity of the two IgG pools at 3 dilutions. FIG. 4A shows that as with whole sera, purified IgG also caused processing inhibition and trapping. Although antibodies against reduced and alkylated protein tested positive on ELISA these antibodies tested negative on the GIA and processing assay. It has been previously reported that a majority of the inhibitory epitopes on recombinant AMA-1 were disulphide bond dependent (Dutta et al., 2002, supra; Anders et al, 1993, Vaccine 16, 40-247); the same appears to be the case with processing inhibition and trapping.

TABLE 1

| IgG sample | ELISA Titer | GIA at 3 dilutions | | |
|---|---|---|---|---|
| 3.5 mg/ml | (OD405 = 1) | 10 | 100 | 1000 |
| Ref-AMA-1 | 116,539 | 71% | 10% | −1% |
| R/A AMA-1 | 44,483 | 4% | 5% | 1% |

Monovalent Fab fragments of inhibitory IgG failed to show trapping (FIG. 4B). Equimolar concentration of intact anti-AMA-1 IgG showed high level of trapping, indicating that antigen cross-linking may be important for trapping. The 66 kDa form accumulated in the presence of high concentration of Fab fragments (FIG. 4B, lanes 4 & 5), similar to the observation with intact IgG. To determine the effect of Fab fragments on PfAMA-1₂₀ processing, 1:2500 dilution of the AMA-1 serum pool was added to the assay as a trapping agent (this dilution efficiently trapped but showed no inhibition of PfAMA-1₆₆ or PfAMA-1₅₂ processing). As observed with intact IgG, PfAMA-1₅₂ appeared only at high Fab concentration (FIG. 4B, lane 4) while at lower concentrations PfAMA-1₄₈₊₄₄ was seen. The intensities of the trapped PfAMA-1₅₂ and PfAMA-1₄₈₊₄₄ were much lower than observed with intact IgG, probably due to competition between Fabs (non-trapping) and the intact antibodies (trapping). In a comparative GIA, IgG at 0.37 mg/ml showed 81% inhibition while purified Fab fragments of the same IgG sample (at 0.28 mg/ml; approximate equimolar antigen binding sites) showed 78% inhibition of parasite invasion. Control IgG and Fab fragments showed no inhibition. The anti-AMA-1 Fab preparation used in the GIA showed a profile similar to FIG. 4B, lanes 4 & 5, while the Fab fragments of pre-immune IgG appeared similar to the control lane-c on a processing assay. The inhibition of invasion by Fab fragments is consistent with the previous observations with P. knowlesi (Thomas et al., 1984, Mol. Biochem. Parasitol. 13, 187-199).

### EXAMPLE 3

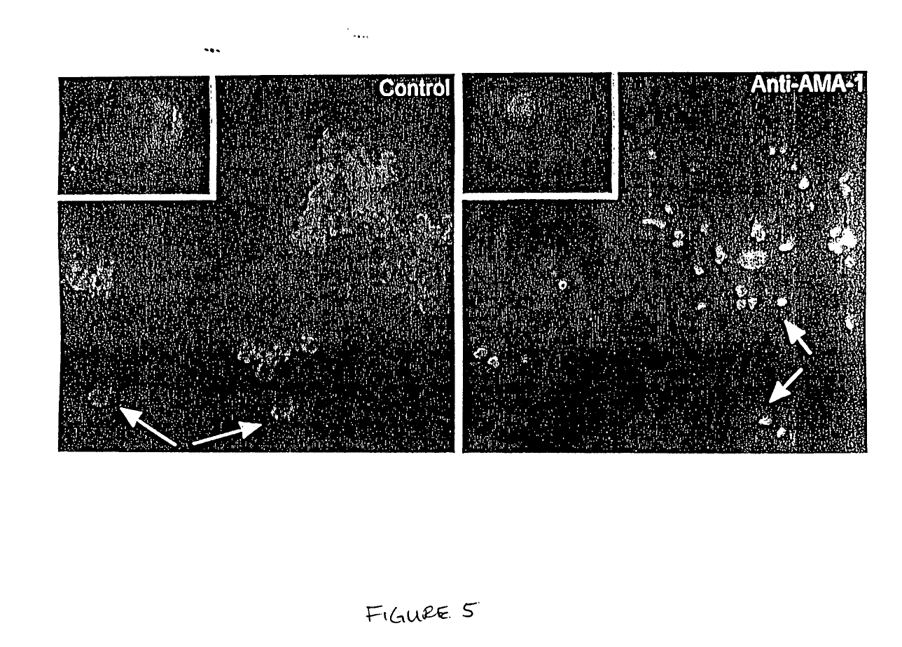

AMA-1 localization assay. AMA-1 was located apically and circumferentially on merozoites released in the presence of pre-immune control pool (1:10 dilution). This is the expected location of AMA-1 on free merozoites (FIG. 5; control). In contrast, AMA-1 on the merozoites released in the presence of immune pool (1:10 dilution) was located apically with little or no circumferential distribution (FIG. 5; Anti-AMA-1). This effect was clearly seen up to 1:1000 dilution of the serum pool. No apical restriction was seen in the presence of inhibitory concentrations of Fab fragments. Hence it appears that apical restriction was associated with cross-linking and trapping.

### EXAMPLE 4

Processing of AMA-1 in the presence of protease inhibitors and cation chelators. Inhibitors of serine proteases (antipain, PMSF, TLCK, TPCK, leupeptin and chymostatin), cysteine proteases (antipain, leupeptin, chymostatin, E64), cation dependent proteases (1,10-phenanthroline, EDTA and EGTA) and asparatic proteases (pepstatin) were used to determine the nature of proteases involved in AMA-1 processing. The assay was also performed in the presence of trapping antibodies (1:2500 dilution of rabbit anti-AMA-1 sera pool). The ability of these protease inhibitors to block schizont rupture or RBC invasion was studied in parallel experiments. Inhibitors 1,10-phenanthroline, TPCK, TLCK and PMSF interrupted schizont maturation, as observed by the presence of mid-stage schizonts on giemsa stained thin smears. PCM's were observed on giemsa stained slides of E64, chymostatin and leupeptin. In the AMA-1 processing assay none of the inhibitors showed significant accumulation of the PfAMA-1₈₃ precursor accompanied by reduced PfAMA-1₆₆ (Panel-a on FIG. 6A). PfAMA-1₆₆ however, was found to accumulate in the presence of chymostatin, with corresponding decrease in the intensity of trapped PfAMA-1₅₂ and PfAMA-1₄₈₊₄₄ (Panel-b on FIG. 6A, lane 5 & FIG. 6B). Chymostatin did not block schizont development however, PCM's were observed on giemsa stained smear. Chymostatin at 100 μM showed >90% inhibition of merozoite invasion. Cation chelating agents EDTA and EGTA also caused accumulation of PfAMA-166 (Panel-a on FIG. 6A, lane 10, 11; FIG. 6B), however, unlike chymostatin where the formation of both PfAMA-1₅₂ and PfAMA-1₄₈₊₄₄ were inhibited, EDTA and EGTA inhibited the formation of only PfAMA-1₄₈₊₄₄ (Panel-b on FIG. 6A, lane 10, 11 & FIG. 6B). EDTA and EGTA did not affect schizont rupture at 1 mM, but showed ˜40% and ˜20% inhibition of RBC invasion respectively. Addition of Ca⁺² to both EDTA and EGTA lanes reversed the accumulation of PfAMA-1₆₆, accompanied by increase in the level of PfAMA-1₄₈₊₄₄ (Panel-a & -b on FIG. 6C). While the addition of Mg⁺² reversed the EDTA induced inhibition, it had no effect on the EGTA induced inhibition (EGTA is a poor chelator of Mg⁺²). We did not observe apical restriction of AMA-1 by IFA on merozoites released in the presence of any of the inhibitors mentioned above.

### EXAMPLE 5

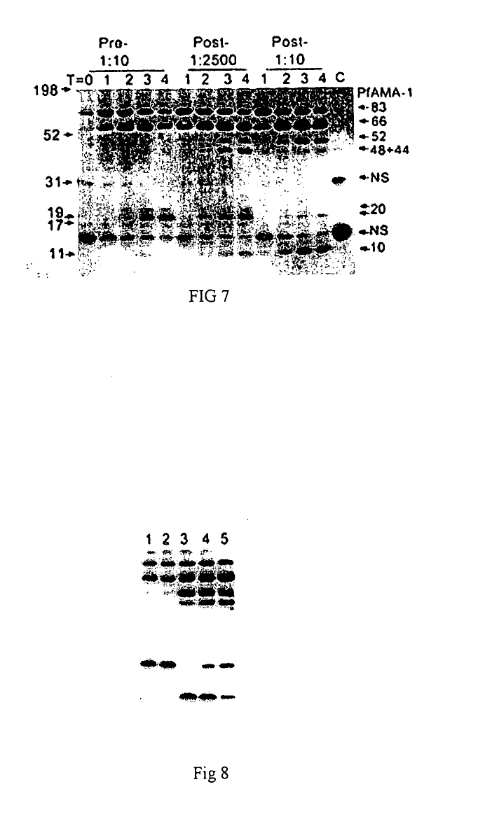

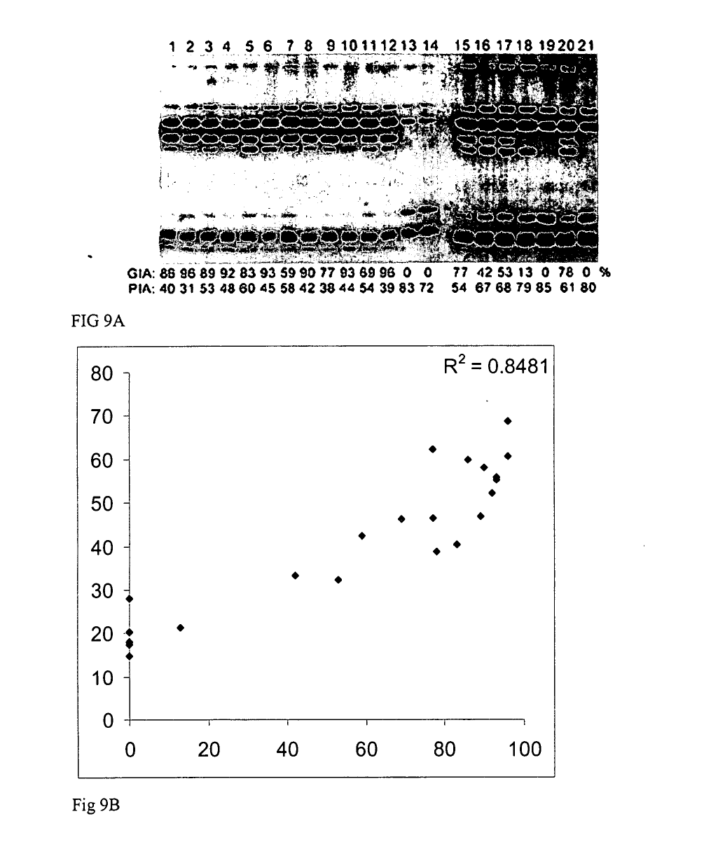

As discussed above, AMA-1 processing is affected by invasion inhibitory antibodies against recombinant AMA-1. FIG. 7 shows the specificity of the AMA-1 processing assay, using a time course experiment whereby AMA-1 synthesis and processing was followed in developing and rupturing schizonts in the presence of either, adjuvant control sera (1:10 dilution, non-inhibitory in a GIA), anti-AMA-1 immune sera (1:2500, non-inhibitory) or immune sera (1:10 dilution, ˜96% inhibition at 1:5 dilution in a GIA). In the presence of control sera, AMA-1 was first detected as PfAMA-1₈₃ (T₀), followed by the appearance of a merozoite bound PfAMA-1₆₆ (T₁-T₄). As merozoite development progressed, the intensity of PfAMA-1₆₆ band decreased and a doublet at ˜20 kDa appeared (intensity of the lower band was much stronger). This doublet reacted only with the C-terminus specific mAb 28G2dc1 and not with the polyclonal anti-AMA-1 ectodomain antibodies (data not shown). Narum & Thomas (1994, supra) have reported that 28G2dc1 reacts with ring stage parasite and Howell et al. (2003, supra) have reported immunprecipitating a doublet of 22-24 kDa from ring stages using 28G2dc1 mAb. The major ˜20 kDa AMA-1 specific band (PfAMA-1₂) seen in our PIA's, represents the membrane bound remnant of the normal PfAMA-1₆₆ processing left over after the shedding of PfAMA-1_{48/44} from the merozoites. In the presence of non-inhibitory concentration of the post-immune sera (1:2500), the PfAMA-1₅₂ and the co-migrating PfAMA-1₄₈₊₄₄ were seen (T₂-T₄) trapped on the merozoites. Two additional bands one corresponding to the PfAMA-1₂₀ a ˜10 kDa was seen. This AMA-1 specific, 10 kDa band (PfAMA-1₁₀), is the membrane bound remnant of PfAMA-1₅₂ cleavage and this band also reacted only with the mAb 28G2dc1 and not with the polyclonal ectodomain antibodies (not shown). At the final time point T₄ in the 1:10 inhibitory serum lane the combined intensities of the PfAMA-1₅₂+PfAMA-1₁₀ bands was higher than the combined intensities of PfAMA-1₄₈₊₄₄+PfAMA-1₂₀ bands, seen in the 1:2500 dilution lane (non-inhibitory). This result agrees with our previous findings that at inhibitory concentrations anti-AMA-1 polyclonal antibodies inhibit the processing of PfAMA-1₆₆ to soluble forms and instead it is cleaved at an alternative site giving rise to a trapped PfAMA-1₅₂ product. Another strong band at ˜15 kDa band was also present on the blot, its intensity decreased as erythrocyte rupture progressed (T₀ to T₄). This band represents the non-specific reaction of the chemiluminescent substrate used in the assay (probably with the iron component in the hemoglobin monomer), lane C has approximately equivalent number of uninfected erythrocytes incubated with 1:10 dilution of immune serum showing that the ˜15 kDa band is indeed erythrocyte derived; another non-specific band at ˜30 kDa was also observed in this control lane, C. In order to further confirm the precursor-product relationship between the PfAMA-1 fragments, serial dilutions 1:10, 1:100 and 1:1000 of the adjuvant control and immune rabbit serum (same sera as in FIG. 7), was tested in a PIA. FIG. 8 shows that AMA-1 processing inhibition is indeed dose dependent. FIG. 8 also shows normal processing of PfAMA-1, in the 1:10 and 1:100 control serum dilutions, similar to the negative rabbit serum control lane (not shown). In the serial dilution of the immune serum, the relative intensities of the PfAMA-1₄₈₊₄₄+PfAMA-1₂₀ bands increases with decreasing serum concentration, while PfAMA-1₅₂+PfAMA-1₁₀ band intensity decreases.

### EXAMPLE 6

To determine the relationship between AMA-1 processing inhibition and invasion inhibition, PIA experiments were carried out with serum from rabbits immunized with recombinant AMA-1. These sera exhibited GIA activities ranging from 0-96% at 1:5 dilution. PIA's were carried out at 1:10, 1:100 and 1:1000 dilution, against the 3D7 strain of P. falciparum. Blots were scanned and band intensities of the 66, 52, 48+44, 20 and 10 kDa forms were compared. Band intensity ratios of various band pairs were plotted against the percent invasion in a GIA and correlation coefficients (R²) were used as the measures of best fit. It was found that PIA activity calculated as percentage intensity of PfAMA-1₁₀/(PfAMA-1₁₀+PfAMA-1₂₀1) at a serum dilution of 1:100 gave the best and most reproducible correlation with the GIA. Hence, we propose that for rabbit serum with high titer anti-AMA-1 antibodies this method of calculating PIA is optimal for the prediction of their GIA activity. Using this method the GIA vs PIA plots obtained from two independent experiments are presented.

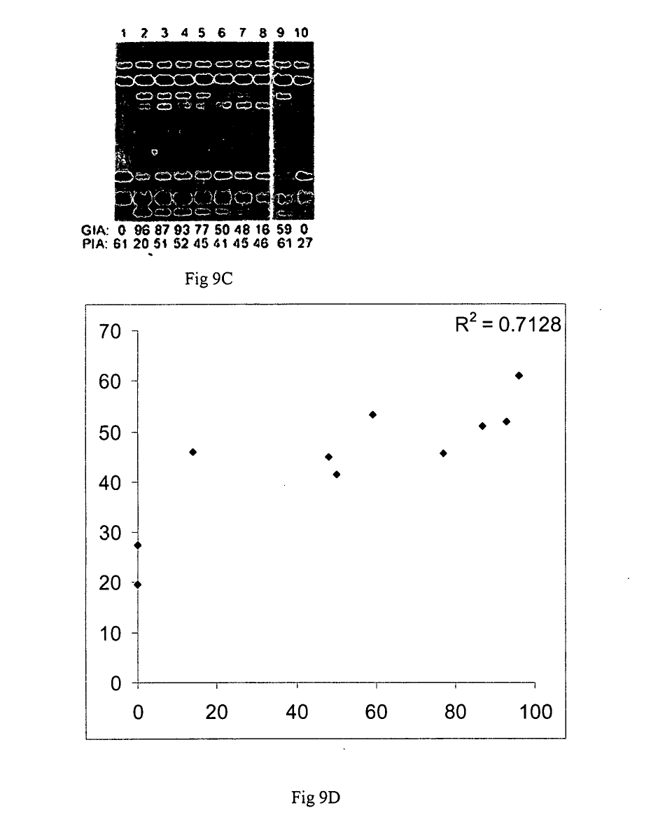

Experiment 1. Sera from animals immunized with 3D7 AMA-1 (2 rabbits) or FVO AMA-1 (#3) or a mixture of 3D7 and FVO AMA-1 proteins (#8) along with adjuvant, an adjuvant control (#1) and a pre-immune serum control (1 rabbit) were used. An IgG fraction from sera of rabbits immunized with refolded 3D7 AMA-1 (inhibitory) and reduced and alkylated AMA-1 (non-inhibitory) along with adjuvant and a no rabbit serum control are also included in the analysis shown in FIG. 9A. FIG. 9B shows s positive correlation between GIA and PIA (R²˜0.8). ELISA end-point titers determined as the dilution that resulted in an OD₄₁₅=1.0 with 3D7 AMA-1 coated on plates. The correlation between ELISA-PIA and ELISA-GIA was lower, ˜0.4 (plot not shown). Experiment 2: Sera from rabbits immunized with 3D7 AMA-1 (4 rabbits), FVO AMA-1 (#1) along with adjuvant, 3D7 AMA-1 (3 rabbits) immunized with adjuvant, an adjuvant control and a no rabbit serum control (FIG. 9C). A positive correlation was observed (R²˜0.7) between GIA and PIA (FIG. 9D).

Discussion:

We have discovered that that antibodies against AMA-1 that inhibit parasite invasion in vitro also inhibit the normal processing of PfAMA-1₆₆ to PfAMA-1₄₈₊₄₄ & PfAMA-1₂₀. Polyclonal antibodies at inhibitory concentrations can additionally cause anomalous processing of the accumulated PfAMA-1₆₆ to a merozoite bound PfAMA-1₅₂ and PfAMA-1₁₀. This assay can also be used to determine the PIA activity of anti-AMA-1 immune reagents such as monoclonal antibodies and Fab fragments, that do not cross-link the 52 and 48+44 kDa bands on merozoites. The assay is sensitive for strain specificity of anti-AMA-1 seen in a GIA. A similar assay can predict AMA-1 based protection in immunized animals and in human volunteers. The amount of PfAMA-1₄₈₊₄₄ band shed into the culture supernatant was also analyzed by western blot (not shown). The controls and low GIA sera had the highest PfAMA-1₄₈₊₄₄ secreted into the supernatant, while reduced quantities were shed by merozoites released in the presence of highly inhibitory sera.

We propose that inhibitory anti-AMA-1 immune reagents as well as protease inhibitors such as EDTA and chymostatin, inhibit invasion by blocking the further processing of PfAMA-1₆₆ to PfAMA-1₄₈₊₄₄. Apical restriction of PfAMA-1₆₆ caused by polyclonal antibodies appears to be related to the appearance of the PfAMA-1₅₂ band. It is possible that the enzyme responsible for the anomalous processing of PfAMA-1₆₆ to PfAMA-1₅₂ and PfAMA-1₁₀ is also located apically on the merozoites. We were unable to inhibit the protease step that generates PfAMA-1₅₂ in the presence of inhibitory anti-AMA-1, indicating that either this cleavage site is not accessible to the protease inhibitors used, considering that Howell et. al. have suggested that it results from a cleavage within the trans-membrane domain or this may be a result of non-specific proteolysis of the apically restricted AMA-1. We had suggested previously that there appears to be a precursor-product relationship between the PfAMA-1₅₂ band and the PfAMA-1₄₈₊₄₄, by observing the band intensities on western blots with merozoites released in the presence of serial dilution of an inhibitory sera. However, our data now shows that PfAMA-1₅₂ is only seen in the presence of cross-linking polyclonal antibodies to AMA-1, moreover, immunostaining PIA blots with 28G2dc1 along with the polyclonal antibodies, no further breakdown of the PfAMA-1₅₂ to PfAMA-1₄₈₊₄₄ was apparent. Hence, we conclude that PfAMA-1₅₂ is a product of anomalous processing and not a normal intermediate of PfAMA-1 processing as proposed earlier

1. A method for detecting anti-AMA-1 antibodies that inhibit AMA-1 processing in a sample comprising:
incubating the sample with P. falciparum parasites and quantifying the formation of one or more AMA-1 processing products.

2. The method of claim 1 wherein said processing intermediate is chosen from the group consisting of PfAMA-1₆₆, PfAMA-1₅₂, PfAMA-1₄₄₊₄₈, PfAMA-1₂₀, and PfAMA-1₁₀.

3. A method for determining the efficacy of an AMA-1 vaccine in producing anti-AMA-1 processing-inhibitory antibodies, said method comprising administering an immunogenic composition comprising AMA-1 or an immunogenic fragment thereof to a subject, obtaining a sample from said subject, and administering the method of claim 1.

4. An immunogenic composition which induces the formation of antibodies that inhibit the processing of AMA-1 in a subject.

5. The composition of claim 4 wherein said composition is AMA-1 or an immunogenic fragment thereof.

6. A method for screening protease inhibitors that inhibit PfAMA-1 processing in Plasmodium falciparum.

7. A kit for determining the presence of processing inhibitory anti-AMA-1 antibodies in a biological sample, comprising:
(i) buffers or components necessary for parasite growth and analysis of parasitic growth stage;
(ii) sera or a combination of antibodies known to inhibit processing to serve as a positive control and sera or a combination of antibodies known to not inhibit AMA-1 processing to serve as a negative control, and buffers or components necessary for dissolving or diluting these factors.
(iii) buffers and components necessary for analyzing the immune complexes;
(iv) means for detecting the immune complexes formed, such as labeled secondary antibodies
(iv) possibly also including an automated scanning and interpretation device for analyzing and quantifying the immune complexes.
